(19) United States
(12) Patent Application Publication
(10) Pub. No.: US 2018/0085485 A1
(43) Pub. Date: Mar. 29, 2018

(54) Electrical Aroma-Dispensing Device

(71) Applicants: Eli Probst, Thousand Oaks, CA (US); Todd Metlen, Ojai, CA (US)

(72) Inventors: Eli Probst, Thousand Oaks, CA (US); Todd Metlen, Ojai, CA (US)

(21) Appl. No.: 15/275,155

(22) Filed: Sep. 23, 2016

(51) Int. Cl. A61L 9/03; B60H 3/00

(52) U.S. Cl. CPC A61L 9/032; B60H 3/0007; B60H 2003/005; A61L 2209/12; B60H 2003/0042; A61L 2209/134

(57) ABSTRACT

An electrical aroma-dispensing device for the purpose of dispensing aromatic scents and substances that includes a connector portion on one end of an outer enclosure for connecting to a power source, an inner cavity, an electrical circuit board inside the inner cavity that includes a current regulator and micro-processor, an internal heating element that can be operationally controlled by the micro-processor, a metal cavity adjacent to the heating element, a substrate material infused with or otherwise containing aromatic oil or liquid that fits into the metal cavity, one or more apertures in the outer enclosure that allow for the passage of air into or out from the interior cavity, a cap covering the substrate material with one more apertures, an internal fan that can be operationally controlled by the microprocessor and directs air through the device towards the metal cavity and substrate material and then out of the device, and a mechanically operated dampener, which may be incorporated into the cap or may be an independent part, with one or more apertures and that can be adjusted to effect the flow air into or out of the device.

### CROSS-REFERENCE TO RELATED APPLICATION

Not-applicable

### STATEMENT REGARDING FEDERALLY SPONSORED RESEARCH OR DEVELOPMENT

Not-applicable

### REFERENCE TO SEQUENCE LISTING, A TABLE, OR A COMPUTER PROGRAM LISTING COMPACT DISC APPENDIX

Not-applicable

### BACKGROUND OF INVENTION

### 1. Technical Field

The present invention relates generally to an electrical aroma-dispensing device for the purpose of dispensing aromatic scents and substances, and relates more specifically to an electric device intended to dispensing aromatic scents and substances in small, enclosed spaces such as automobiles and interior rooms.

### 2. Prior Art

There are other aromatic articles and electric devices that do not include the novel improvements of the invention disclosed herein. Generally the purpose of an aromatic device is to release a desired scent into the atmosphere to fill an environment with a particular fragrance or obscure an existing scent that is already present in the environment. Many such articles and devices intended for use in interior spaces, such as automobiles and homes, are well known. There are several environmental factors that can adversely effect the efficacy of such articles and devices, such as temperature and airflow.

Some articles and devices are comprised of a fragrant material substrate or substance fully or partially exposed to the atmosphere. U.S. Pat. No. 7,770,817 discloses a portable air scenting device which comprises at least one material used in the manufacture of an interior part of a new transportation Vehicle such as an automobile. The material has at least one; scent, and the material is prepared and formed to have an enhanced surface exposure to enhance the at least one scent. Unfortunately, this prior art example does not include an active method for enhancing the release of fragrance such as heat and there are no methods included for effecting the amount of air exposed to fragrance, or for mitigating the potential influence of environmental conditions, such as temperature and airflow, on the effective release of fragrance into the atmosphere.

The device described in U.S. Pat. No. 7,955,552 A car freshener system includes a portable reservoir seated within the vehicle, a predetermined volume of a liquid air freshening compound housed Within the reservoir, and a portable discharge casing disposed within the vehicle with a rotary discharging mechanism housed therein. The discharge casing includes an input port and an output port in communication with the reservoir in such a manner that the liquid air freshening compound is caused to enter through the input port and travels along a unidirectional accurate path along a major interior circumference of the discharge casing prior to exiting through the output port. Unfortunately, this prior art example does not include a method for controlling or effecting the amount of air exposed to fragrance, or for mitigating the potential influence of environmental conditions, such as temperature and airflow, on the effective release of fragrance into the atmosphere.

There are other devices that actively operate to dispense fragrance. U.S. Pat. No. 6,592,828 discloses a rechargeable electric air freshener for vehicles of a type that is placed in a lighter holder of these vehicles includes a case, a lid for closing the case, each provided with openings for the perfuming vapor from an aromatic substance, which may be recharged and which, by means of temperature, releases aromatic essences it contains, situated inside the case, two metal bands on sides of the case to hold the ease in position, one permitting connection to one electrical pole, a button at a rear portion of the case, a spring inside the case for the other pole, an integrated printed heated electrical circuit in the case, and an external button with four positions for activating the electrical circuit, the external button including an LED visible behind the lid and a switch, which activates a sound alarm to advise of a need to recharge the aromatic substance. Unfortunately, this prior art example does not includes a method for controlling or limiting the amount of air exposed to fragrance or for mitigating the possible influence of environmental airflow conditions on the effective release of fragrance into the atmosphere.

The invention described in U.S. Pat. No. 8,750,694 discloses an automobile 12 volt air freshener that includes a connecter portion and a head portion, the connecter portion interconnectable with an automobile cigarette lighter, the head portion rotatably, anglably, and extensibly moveable upon a shaft disposed on a proximal end of the connector portion, the head portion including a bulb-shaped casing and an interior cavity, the head portion having a thermostat disposed within the interior cavity, the thermostat in operational communication with a heating element, with a fill reservoir releasably attachable to a reservoir input by means of a threaded fastener, whereby an aromatic scent disposed within the fill reservoir is conveyed to a sorbent member within the interior cavity, and the aromatic scent is volatilized when the heating element is activated. Unfortunately, this prior art example offers no method to increase or decrease the amount of air exposed to fragrance or mitigate the potential influence of environmental airflow conditions on the effective release of fragrance into the atmosphere.

Accordingly, the present invention is disclosed in order to overcome the above noted shortcomings. The electrical aroma-dispensing device disclosed herein is convenient and easy to use, mitigates the most common environmental conditions that prevent effective discharge of a scent, is user-adjustable but not dependent, and is user-refillable. An important advantage of this electric aroma-dispensing device is that it incorporates a user controllable dampener to control the airflow into and from the device, as well as both an active heat element and an internal fan, which are controlled by a micro-processor and are capable of working both separately and in conjunction, to achieve superior and longer-lasting output of aroma in a variety of environmental conditions when compared to other devices that are currently available.

### BRIEF SUMMARY OF INVENTION

In view of the foregoing background, it is therefore an object of the present invention to provide an apparatus for effectively discharging a scent inside an interior location, such as a room or automobile. These and other objects, features, and advantages of the invention are provided by the electrical aroma-dispensing device.

The electrical aroma-dispensing device includes a connector incorporated into the outer enclosure for connecting the device to a power source. In one embodiment, the connector portion is a USB style connector designed to interface with a 5V DC power source. In another embodiment, the connector portion is configured to reliably fit into an automobile 12V DC cigarette lighter outlet and includes a connector body with a distal end.

The connector portion is electrically coupled to a circuit board, which includes a current regulating device, a micro-processing unit for controlling and vary the operating status of an internal heating element and/or an internal fan, and one or more LEDs to provide the user feedback regarding the current operational status of the device.

The LED(s) continuously illuminate when the device is connected to a power source and the internal heating element and/or internal fan in operation. The LED flashes intermittently when the device is connected to power, but neither the heating element nor the fan is operating. The LED does not illuminate when the device is not connected to a power source.

The circuit board is electrically connected to an internal fan and an internal heating coil. When activated, the fan circulates air drawn through one or more aperture in the outer enclosure and directs that air through the inside of the device towards a fragrant substrate material placed inside a user accessible metal cavity. One or more apertures in the outer enclosure situated near the metal cavity allow air directed by the fan through the device to pass through and/or over the substrate material portion and then exit the device. The material substrate may be an open-cell or porous material that allows air directed by the fan to more easily pass through it. The heating element portion is located directly under or adjacent to the metal cavity for holding the fragrant substrate material. When activated, the heating element causes the cavity to increase in temperature and heat the substrate material to improve the volatility and facilitate the diffusion of any aromatic liquids or oils into the environment. The metal cavity for the substrate material is covered by a removable cap containing one or more apertures that can be manually adjusted to control the passage of air and fragrance from the device into the external atmosphere.

Covering the aperture(s) in the outer enclosure is a dampener, which may be incorporated in to the cap or may be an independent part, that contains one more apertures and is manually adjusted to designed to effect the passage of air through the apertures into and/or out of the outer enclosure of the device. This allows the user to control the flow of air into and/or out of the device and effectively adjust the amount of fragrance released into the environment.

### BRIEF DESCRIPTION OF THE DRAWING FIGURES

### Figures

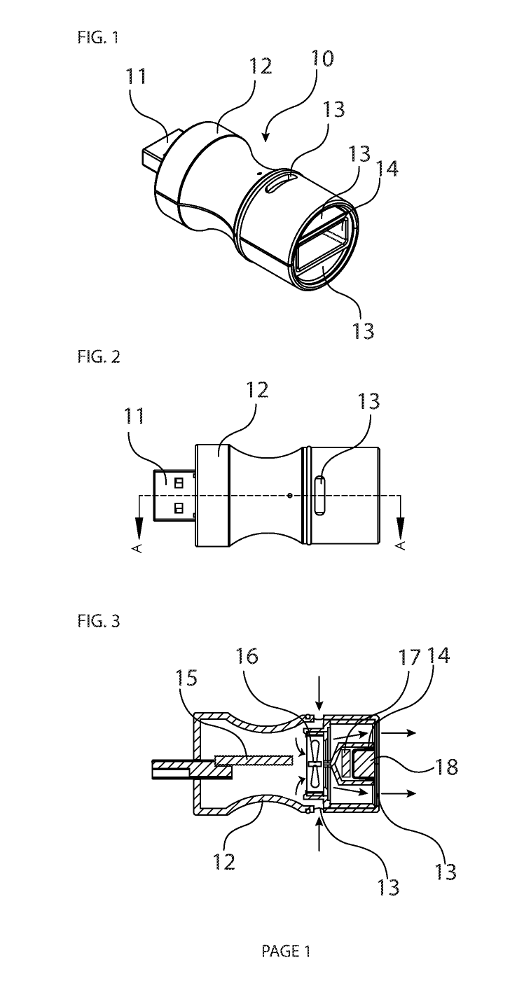

FIG. 1 is an isometric view of one embodiment of the present invention

FIG. 2 is a top view of one embodiment of the present invention

FIG. 3 is a cross-section view taken along line A-A of FIG. 2 of an embodiment of the present invention that illustrates the airflow through the device past the substrate material

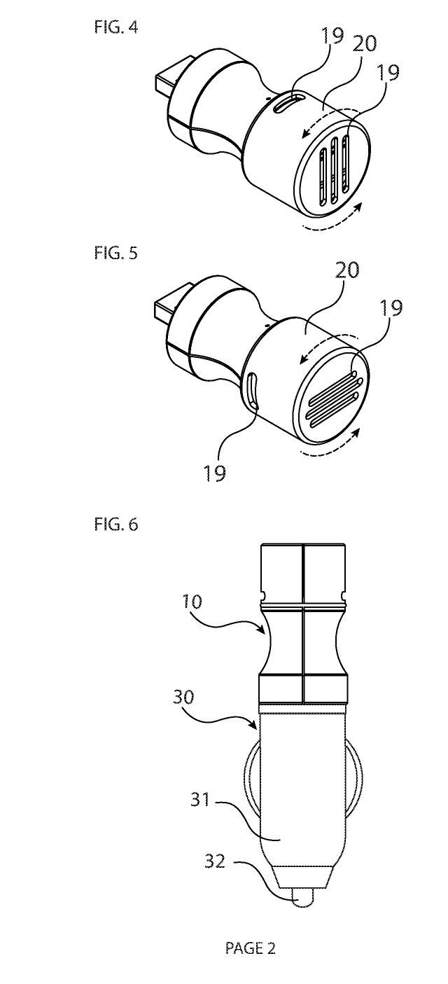

FIG. 4 is an isometric view of an embodiment of the current invention illustrating the dampener/cap positioned with apertures in alignment

FIG. 5 is an isometric view of an embodiment of the current invention illustrating the dampener/cap positioned with apertures out of alignment

FIG. 6 is a side view of an embodiment of the current invention illustrating a 12V DC connector that includes a connector portion and a distal end.

### DETAILED DESCRIPTION OF THE INVENTION

The following detailed description is of a preferred embodiment of the present invention, but is not to be taken in a limiting sense. The following detailed description is made merely for illustrating the general principles of the present invention, since the scope of the invention is best defined by the claims.

Referring to FIGS. 1-5, an embodiment of the 5V DC electrical aroma dispensing devise 10 is illustrated.

The electrical aroma dispensing device includes a connector portion, which may be a standard Type A USB connector 11 for connecting to a 5V DC power source. The connector portion is attached to the outer enclosure 12.

The outer enclosure 12 includes one or more apertures 13 that allow for air to pass through the enclosure into and/or out of the inner cavity of the enclosure 12. On the end of outer enclosure 12 opposite the connector 11 is the cavity 14 for aromatic liquid or substrate 17 containing an aromatic liquid. The outer enclosure 12 also contains a circuit board 15 with a micro-processor electrically connected to the heating element 17 and/or the internal fan 16, which cycles the heating element 17 and/or internal fan 16 on and off according to a pre-programmed algorithm.

As the air passes through the outer enclosure 12, an internal fan 16 directs the air through the interior of the device towards the metal cavity 14, situated adjacent to a heating element 17 and containing a substrate 18 that may be infused with or otherwise contain an aromatic liquid or oil, and then out of the device through one or more aperture(s) 13.

Covering part of the outer enclosure 12 is a mechanically operated dampener/cap 20 with one or more apertures 19 that may be positioned over the aperture(s) 13 in the outer enclosure 12. FIG. 4 illustrates that the dampener/cap's aperture(s) 19 may be aligned with the outer enclosure's apertures 13 to allow for the unobstructed passage of air into and/or out of the interior of the outer enclosure 12. FIG. 5 illustrates that the dampener/cap's aperture(s) 19 may be misaligned with the outer enclosure's apertures 13 to partially or fully obstruct the passage of air into and/or out of the interior of the outer enclosure 12.

FIG. 6 illustrates an embodiment of the electrical aroma dispensing device 10 where it's electrical connector 30 includes a connector body 31 and a distal end 32 designed to interface with a 12V automobile cigarette lighter/power outlet.

What is claimed:

1. An electrical aroma-dispensing device comprising:
a connector portion on the exterior housing for connecting to a 5V DC power source
a cavity, made of a metal material, for placement of a substrate material that may be infused with or otherwise contain an aromatic liquid or oil so as to be made fragrant
a heating element disposed in an interior cavity located directly adjacent to or below the metal cavity intended for the placement of a substrate material
a fan disposed in an interior cavity that directs air through the device and towards the metal cavity intended for the placement of a substrate material
an electrical circuit board containing a current regulating device and a micro-processor that is electrically connected to and controls the operating status of the heating element and/or the fan according to a pre-programmed algorithm
an outer enclosure with one or more apertures that allow for air to pass through the outer enclosure into and/or out of the interior of the outer enclosure
a removable cap covering the substrate material that contains one or more apertures and that can be manually adjusted to control the passage of air and fragrance from the device into the external atmosphere.
a manually operated dampener, which may be incorporated into the removable cap or may be a independent part, that can be adjusted to control the passage of air into and/or out of the apertures in the outer enclosure

2. The electrical aroma-dispensing device of claim 1 that includes a substrate made of an open-cell or porous material infused with or otherwise containing an aromatic liquid or oil.

3. The electrical aroma-dispensing device of claim 1 that includes a user-removable or replaceable material substrate infused with or otherwise containing an aromatic liquid or oil.

4. The electrical aroma-dispensing device of claim 1 that includes a cap or cover with one or more apertures that retains a substrate inside the cavity.

5. The electrical aroma-dispensing device of claim 1 in which the heat element is capable of progressively increasing in temperature in correspondence with the increase in time of its continuous active operation.

6. The electrical aroma-dispensing device of claim 1 that contains a temperature sensor to provide feedback to the circuit regarding environmental temperature conditions such that the heat element and/or internal fan can be activated/deactivated automatically by the circuit as determined by a program.

7. The electrical aroma-dispensing device of claim 1 that contains one or more light emitting diodes (LEDs) that provide feedback to the user regarding the operational status of the device.

8. An electrical aroma-dispensing device comprising:
a connector portion on the exterior housing for connecting to a 12V DC power source
a cavity, made of a metal material, for placement of a substrate material that may be infused with or otherwise contain an aromatic liquid or oil so as to be made fragrant
a heating element disposed in an interior cavity located directly adjacent to or below the metal cavity intended for the placement of a substrate material
a fan disposed in an interior cavity that directs air through the device and towards the metal cavity intended for the placement of a substrate material
an electrical circuit board containing a current regulating device and a micro-processor that is electrically connected to and controls the operating status of the heating element and/or the fan according to a pre-programmed algorithm
an outer enclosure with one or more apertures that allow for air to pass through the outer enclosure into and/or out of the interior of the outer enclosure
a removable cap covering the substrate material that contains one or more apertures and that can be manually adjusted to control the passage of air and fragrance from the device into the external atmosphere.
a manually operated dampener, which may be incorporated into the removable cap or may be a independent part, that can be adjusted to control the passage of air into and/or out of the apertures in the outer enclosure

9. The electrical aroma-dispensing device of claim 8 that includes a substrate made of an open-cell or porous material infused with or otherwise containing an aromatic liquid or oil.

10. The electrical aroma-dispensing device of claim 8 that includes a user-removable or replaceable material substrate infused with or otherwise containing an aromatic liquid or oil.

11. The electrical aroma-dispensing device of claim 8 that includes a cap or cover with one or more apertures that retains a substrate inside the cavity.

12. The electrical aroma-dispensing device of claim 8 in which the heat element is capable of progressively increasing in temperature in correspondence with the increase in time of its continuous active operation.

13. The electrical aroma-dispensing device of claim 8 that contains a temperature sensor to provide feedback to the circuit regarding environmental temperature conditions such that the heat element and/or internal fan can be activated/deactivated automatically by the circuit as determined by a program.

14. The electrical aroma-dispensing device of claim 8 that contains one or more light emitting diodes (LEDs) that provide feedback to the user regarding the operational status of the device.
